(19) United States
(12) Patent Application Publication
(10) Pub. No.: US 2026/0335538 A1
(43) Pub. Date: Oct. 29, 2026

(54) AI-GUIDED MODULAR SUBCUTANEOUS BATTLEFIELD DRUG DELIVERY SYSTEM WITH MANUAL, AUTONOMOUS, AND REMOTE-CONTROL CAPABILITIES

(71) Applicant: Jonathan Lopez Diaz, Palm Bay, FL (US)

(72) Inventor: Jonathan Lopez Diaz, Palm Bay, FL (US)

(21) Appl. No.: 19/323,353

(22) Filed: Sep. 9, 2025

Related U.S. Application Data
(60) Provisional application No. 63/795,319, filed on Apr. 26, 2025.

(51) Int. Cl. A61M 5/142; A61M 5/14; A61M 39/24

(52) U.S. Cl. CPC A61M 5/14244; A61M 5/1407; A61M 39/24; A61M 2005/14208; A61M 2005/14252; A61M 2005/14272; A61M 2005/14284; A61M 2202/0007; A61M 2202/04; A61M 2205/10; A61M 2205/121; A61M 2205/3303; A61M 2205/3327; A61M 2205/3331; A61M 2205/3368; A61M 2205/3379; A61M 2205/3553; A61M 2205/3592; A61M 2205/502; A61M 2205/581; A61M 2205/583; A61M 2209/088; A61M 2210/083; A61M 2230/005; A61M 2230/04; A61M 2230/20; A61M 2230/50

(57) ABSTRACT

A modular wearable medical system includes a plurality of subcutaneous delivery modules each secured to a user's limb and fluidly coupled to a pharmaceutical compound cartridge. Each module incorporates a micro-pump, a cannula deployment mechanism, and a cartridge interface with a seal-verification sensor. Physiological sensors continuously monitor biometric parameters, and a control unit determines dosing parameters for autonomous delivery via electronically actuated valves. The system supports manual and remote activation through an encrypted communication interface and displays tactical guidance in response to trauma. Cartridges may include single or multiple compartments formed from biocompatible materials, each having anti-backflow valves and electronically controlled channels. The system incorporates electromagnetic pulse shielding, conformal protective coatings, and secure authentication elements to ensure operational integrity in extreme environments.

### CROSS-REFERENCE TO RELATED APPLICATIONS

The present application claims priority to U.S. Provisional Application No. 63/795,319 filed Apr. 26, 2025, titled “MODULAR AUTONOMOUS BATTLEFIELD MEDICAL SYSTEM FOR REAL-TIME TRAUMA DIAGNOSIS AND AUTOMATED TREATMENT,” which is hereby incorporated by reference in its entirety.

### TECHNICAL FIELD

The embodiments generally relate to the technical field of systems and methods for modular, wearable battlefield medical treatment and drug delivery.

### BACKGROUND

Wearable medical technologies have been developed to assist in monitoring and supporting individuals in challenging environments, including combat and emergency response settings. Conventional devices often include separate platforms for physiological monitoring and for drug administration. Wearable vital sign monitors, for example, may use optical, electrical, or mechanical sensors to collect data such as heart rate, oxygen saturation, and temperature, transmitting these readings to a handheld or remote device for review.

Field drug delivery systems, such as auto-injectors, are designed to administer a predetermined dose of a pharmaceutical compound when activated by the user. These devices typically incorporate a spring-loaded or gas-driven injection mechanism, along with a single-use needle and sealed drug reservoir. Manual activation is generally required, often by pressing the device against the injection site and triggering the release.

Emergency medical equipment such as defibrillators, tourniquet systems, or fluid infusion pumps may also be deployed in operational environments. These devices can provide rapid intervention in critical situations but are generally standalone units, each requiring separate storage, deployment, and operation. While effective in their respective roles, such conventional systems often require the coordination of multiple devices, each with its own activation procedures and operational constraints.

### SUMMARY

This summary is provided to introduce a variety of concepts in a simplified form that is further disclosed in the detailed description of the embodiments. This summary is not intended to identify key or essential inventive concepts of the claimed subject matter, nor is it intended to determine the scope of the claimed subject matter.

In one aspect, the disclosed system integrates physiological monitoring and pharmaceutical compound delivery into a modular, wearable platform that can operate autonomously, under user control, or via remote command. A plurality of subcutaneous delivery modules are configured for secure placement on the body, each including a micro-pump, a cartridge interface with seal verification, and a cannula deployment mechanism. These modules communicate with a control unit that receives continuous biometric data from integrated sensors, analyzes that data, and determines optimal dosing parameters.

In another aspect, the system supports single or multi-compartment cartridges formed from biocompatible, high-barrier materials and equipped with electronically actuated valves to prevent cross-contamination. Each cartridge may be shaped to match anatomical contours and may include anti-backflow valves, embedded authentication features, and protective coatings to ensure operational reliability.

In some variations, the system employs secure wireless communication with adaptive frequency hopping and electromagnetic pulse shielding to maintain function in contested environments. A tactical instruction interface may provide condition-specific visual guidance, enabling users to take complementary non-pharmacological actions during emergencies.

By combining biometric sensing, automated dosing algorithms, secure communication, and modular cartridge-based delivery, the disclosed system addresses the functional separation of conventional monitoring and intervention devices. This integration enables rapid, coordinated, and targeted treatment in high-stress environments without requiring multiple standalone systems.

Other illustrative variations within the scope of the invention will become apparent from the detailed description provided hereinafter. The detailed description and enumerated variations, while disclosing optional variations, are intended for purposes of illustration only and are not intended to limit the scope of the invention.

### BRIEF DESCRIPTION OF THE DRAWINGS

A more complete understanding of the embodiments, and the attendant advantages and features thereof, will be more readily understood by references to the following detailed description when considered in conjunction with the accompanying drawings wherein:

FIG. 1 illustrates a system architecture diagram, according to some embodiments;

FIG. 2 illustrates a schematic block diagram of a modular, wearable battlefield medical system 206 and its associated components, according to some embodiments;

FIG. 3 illustrates a flow diagram representing an operational method of the modular, wearable battlefield medical system, according to some embodiments;

FIG. 4 illustrates a cannula deployment mechanism within the modular, wearable battlefield medical system, according to some embodiments;

FIG. 5 illustrates a control interface that may be implemented as a smartwatch-style device or other wearable unit operatively coupled to the modular, wearable battlefield medical system, according to some embodiments;

FIG. 6 illustrates a communication architecture for the modular, wearable battlefield medical system, according to some embodiments; and

FIG. 7 illustrates a flow diagram of an autonomous decision-making process performed by the modular, wearable battlefield medical system, according to some embodiments.

### DETAILED DESCRIPTION

The specific details of the single embodiment or variety of embodiments described herein are set forth in this application. Any specific details of the embodiments described herein are used for demonstration purposes only, and no unnecessary limitation(s) or inference(s) are to be understood or imputed therefrom.

Before describing exemplary embodiments in detail, it is noted that the embodiments reside primarily in combinations of components related to devices and systems. Accordingly, the device components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

The disclosed system may include one or more modular subcutaneous delivery modules configured for secure placement on a user's upper limb. Each module may be attached using a combination of medical-grade adhesive and an external retention band sized to maintain anatomical conformity. The housing of each module may be formed from a biocompatible material and shaped to match the curvature of the arm, allowing placement in posterior, lateral, or medial orientations without displacement during movement. Surfaces in contact with the skin may comply with ISO 10993 biocompatibility standards.

Each delivery module may contain a micro-pump sized to fit within a housing of approximately 50 mm by 25 mm by 10 mm. The micro-pump may employ a piezoelectric actuator, a micro-solenoid mechanism, or in some embodiments a micro-gear or diaphragm pump. A microcontroller unit (MCU) may coordinate pump actuation, valve control, and communications. A rechargeable battery may supply power to the pump and electronics, while an embedded electromagnetic pulse shield may protect all circuitry from high-energy disturbances.

The delivery module may further include a cartridge interface configured to receive a replaceable pharmaceutical compound cartridge. The interface may include a click-lock coupling to secure the cartridge and a seal-verification sensor to ensure proper alignment before activation. Each cartridge may be constructed from medical-grade high-barrier polymers such as cyclic olefin copolymer (COC), cyclic olefin polymer (COP), or polyether ether ketone (PEEK). Cartridges may include either a single fluid compartment or multiple compartments separated by an inert barrier. In a dual-compartment configuration, each compartment may connect to a separate fluid channel controlled by an electronically actuated microvalve, such as a microelectromechanical system (MEMS) valve or a miniature solenoid valve.

Upon activation, the selected compound may be directed through a dedicated channel to a retractable needle and soft cannula assembly. The needle may deploy to insert the cannula beneath the skin and then withdraw, leaving only the flexible cannula in place for infusion. The cannula may be fabricated from polytetrafluoroethylene (PTFE) or medical-grade polyurethane and may include an anti-kink structure and an anti-reflux valve. In some embodiments, the cannula may retract or seal upon cartridge removal to contain biohazardous material.

Cartridge capacity may range from 0.5 to 1.0 milliliters per compartment for dual-compartment designs, or up to 2.0 milliliters for single-compartment designs. The pump control system may allow precise microdosing, with minimum volumes of approximately 0.25 microliters per pulse. For lipid-based formulations, the output may be limited to 1 to 2 microliters per pulse to prevent tissue saturation, while aqueous formulations may be delivered at rates up to 20 microliters per pulse.

The disclosed system may incorporate multiple biosensor modules configured to measure physiological parameters. These sensors may include electrocardiogram (ECG) electrodes, photoplethysmography (PPG) optical sensors, oscillometric blood pressure sensors implemented as micro-cuffs, skin temperature probes, and electrochemical biosensors for detecting biomarkers such as lactate, cortisol, or glucose. Sensors may be positioned in sweat-contact areas or integrated into medical-grade bracelets worn on the limbs.

Sensor data may be transmitted to a control unit, which may be implemented as a smartwatch-style device worn on the wrist or integrated into tactical gear. The control unit may execute algorithms to analyze real-time biometric data and determine dosing parameters for the delivery modules. An onboard artificial intelligence engine may be trained to detect anomalies such as hemorrhage, dehydration, or cardiac arrhythmias and select appropriate pharmaceutical compounds from available cartridges.

The control unit may also serve as a tactical instruction interface. When triggered by sensor-detected conditions, the interface may display visual or textual guidance such as “apply tourniquet to left arm” or “move to recovery position.” These instructions may be designed to complement pharmacological treatment with behavioral interventions.

Communication between the modules and the control unit may occur through secure short-range wireless links, such as Bluetooth Low Energy (BLE) or software-defined radio (SDR) modules. SDR configurations may enable adaptive frequency hopping, auto-termination of compromised links, and reassignment of channels in the presence of jamming or spoofing attempts. The communication subsystem may also support encrypted long-range links to a remote medical control station, enabling remote monitoring and manual intervention.

Each pump module may contain a hardware-based trusted platform module (TPM) for secure cryptographic operations and tamper detection. Cartridges may incorporate an encrypted near-field communication (NFC) chip to authenticate the cartridge before activation. Physical misconnection may be prevented by using color-coded or mechanically distinct connectors for different compound classes.

System events, including autonomous dosing decisions and manual overrides, may be logged in an encrypted storage system. Local logs may be maintained within each pump's tamper-proof memory, while the control unit may maintain a separate record of user commands and remote override events. Synchronization with remote command interfaces may occur periodically via the secure communication link.

The modular configuration may allow multiple pump modules to be mounted on each limb, each assigned to deliver a specific compound class. In some embodiments, synchronized cannula deployment may be triggered from the control unit at the start of operation, enabling simultaneous readiness across all modules.

Alternative embodiments may vary in the specific actuation mechanism, biosensor placement, communication protocol, or cartridge material selection, provided that the system maintains the ability to monitor biometric data, analyze it, and deliver compounds subcutaneously through interchangeable modules.

The disclosed system provides an integrated solution that combines continuous physiological monitoring, automated analysis, and modular subcutaneous drug delivery into a single wearable platform, unlike conventional systems that rely on separate devices for sensing and treatment. It uses interchangeable compound cartridges, secure wireless communication, and an onboard artificial intelligence engine to autonomously or remotely deliver precisely dosed pharmaceuticals in response to real-time biometric data. This integration enables rapid, coordinated, and context-specific interventions, including both pharmacological delivery and tactical behavioral guidance, without requiring the user to operate multiple independent devices or manually coordinate treatment steps in high-stress environments.

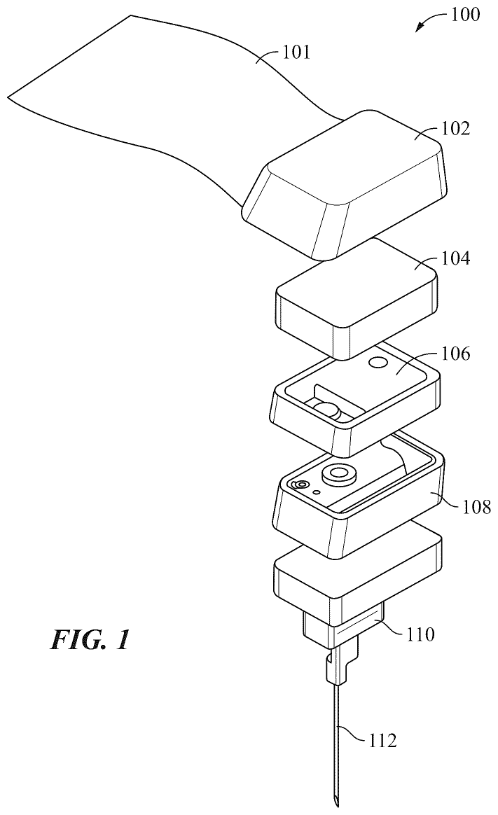

FIG. 1 depicts a variation of the disclosed wearable battlefield medical system 100 including a flexible armband 101 supporting a plurality of independently operable micro-pump units 106. Each micro-pump unit 106 may include an electronically actuated valve assembly 108 and a cartridge interface having a seal-verification sensor 104. The system 100 may include plurality of replaceable compound cartridges 110 shaped to match the anatomical curvature of the arm, each cartridge containing at least one pharmaceutical compound and an integrated one-way anti-backflow valve. Upon activation, a selected compound may be directed through a dedicated channel to a retractable needle 112 and soft cannula assembly. The needle 112 may deploy to insert the cannula beneath the skin and then withdraw, leaving only the flexible cannula in place for infusion.

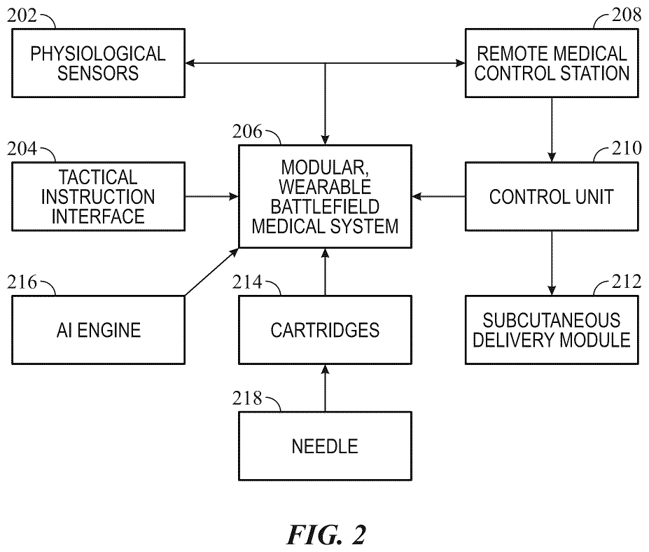

FIG. 2 illustrates a schematic block diagram of a modular, wearable battlefield medical system 206 and its associated components. The system 206 is configured to receive physiological data from one or more physiological sensors 202. The physiological sensors 202 may include electrocardiogram electrodes, photoplethysmography sensors, oscillometric pressure cuffs, temperature probes, or electrochemical biomarker sensors, each configured to continuously monitor biometric parameters of a user.

The system 206 communicates with a tactical instruction interface 204 that is configured to provide real-time visual or textual guidance to the user, such as behavioral or procedural instructions in response to detected physiological events. The system 206 is further operatively coupled to a remote medical control station 208, which may transmit or receive encrypted data and permit remote medical personnel to review sensor information and issue override commands.

A control unit 210 interfaces with the system 206 and provides command and coordination functions. The control unit 210 is operatively connected to one or more subcutaneous delivery modules 212 that may be affixed to the body of the user. Each subcutaneous delivery module 212 is configured to accept one or more cartridges 214, each cartridge housing a pharmaceutical compound or multiple compounds separated by internal barriers.

The cartridges 214 fluidly communicate with a needle 218, which may include a retractable cannula for subcutaneous delivery of the compound. In some embodiments, a seal-verification sensor may be integrated within the cartridge interface to ensure correct alignment prior to delivery. An AI engine 216 is shown in communication with the system 206, where the AI engine 216 is configured to analyze biometric data from the physiological sensors 202, determine anomalies, and autonomously generate dosing commands transmitted through the control unit 210 to the subcutaneous delivery module 212.

In this configuration, the system 206 coordinates data flow between the physiological sensors 202, the tactical instruction interface 204, the AI engine 216, and the remote medical control station 208, while controlling the delivery of compounds through the cartridges 214 and needle 218.

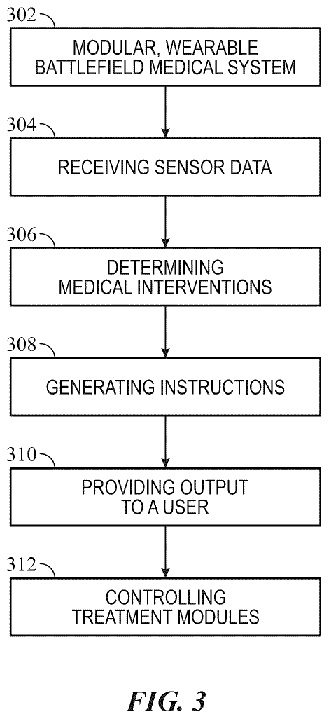

FIG. 3 illustrates a flow diagram representing an operational method of the modular, wearable battlefield medical system 302. At step 304, the system receives sensor data from one or more physiological sensors configured to monitor biometric parameters such as heart rate, blood oxygen saturation, blood pressure, temperature, and biochemical markers. The received data is then processed at step 306, where the system determines medical interventions based on detected physiological conditions or anomalies.

At step 308, the system generates instructions corresponding to the determined interventions. These instructions may include either control signals for compound delivery modules or guidance information for behavioral actions such as applying a tourniquet or adjusting posture. At step 310, the system provides output to a user, for example through a tactical instruction interface or a wearable control device. Finally, at step 312, the system controls one or more treatment modules, such as subcutaneous delivery modules containing compound cartridges, to administer the determined intervention under autonomous, manual, or remote control.

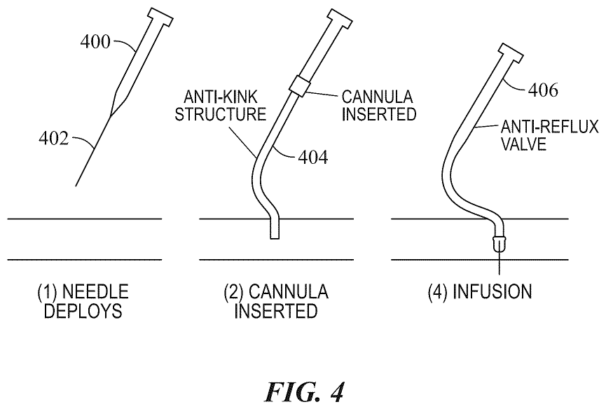

FIG. 4 illustrates a cannula deployment mechanism within the modular, wearable battlefield medical system. In a first stage, a needle 400 is advanced through a skin surface 402 to create an insertion pathway. In a subsequent stage, a cannula 404 is guided into the subcutaneous tissue through the created pathway. The cannula 404 may include an anti-kink structure configured to preserve lumen patency during flexion or movement of the user. Once the cannula 404 is fully positioned, the needle 400 may be retracted, leaving only the cannula in place for compound delivery. In a later stage, the cannula 404 may be coupled with an anti-reflux valve 406 configured to prevent backflow of infused compounds. The final infusion stage permits the controlled administration of pharmaceutical agents through the cannula 404 while maintaining structural integrity and preventing unintended leakage.

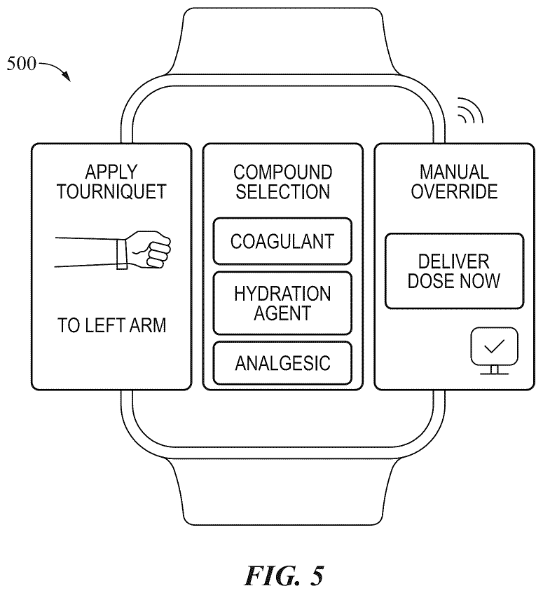

FIG. 5 illustrates a control interface 500 that may be implemented as a smartwatch-style device or other wearable unit operatively coupled to the modular, wearable battlefield medical system. The control interface 500 is configured to display multiple functional screens providing both user guidance and system control options.

A first screen of the control interface 500 displays tactical guidance instructions to the user, for example directing the user to “APPLY TOURNIQUET TO LEFT ARM,” along with a corresponding graphical icon. Such instructions may be generated in response to sensor-detected physiological events and are intended to complement pharmaceutical interventions.

A second screen provides compound selection options, allowing the user or remote medical personnel to view or select among available compounds contained within cartridges. As illustrated, selectable options include a coagulant, a hydration agent, and an analgesic, though additional compound classes may be supported.

A third screen provides a manual override function. The manual override permits the user or remote operator to bypass autonomous system control and directly command administration of a selected compound. As shown, a “DELIVER DOSE NOW” activation element is provided, along with confirmation controls to ensure intentional input.

The control interface 500 may also display indicators of wireless connectivity, system status, or receipt of remote medical commands, ensuring secure and reliable communication between the control unit, delivery modules, and remote medical control stations.

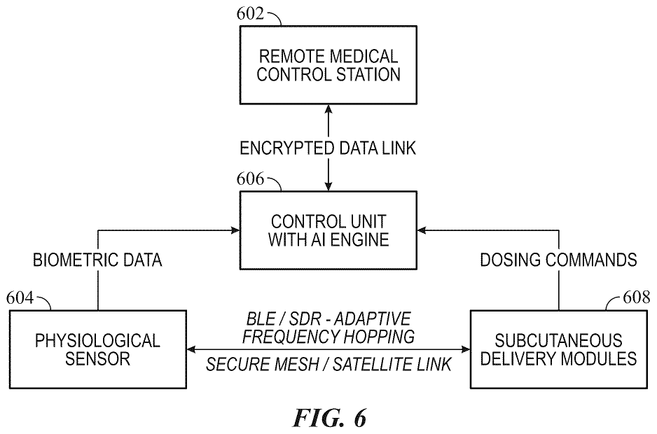

FIG. 6 illustrates a communication architecture for the modular, wearable battlefield medical system. A remote medical control station 602 is shown in encrypted data communication with a control unit 606 that incorporates an artificial intelligence (AI) engine. The encrypted data link permits secure bidirectional transfer of medical information, remote override commands, and treatment logs between the control unit 606 and the remote medical control station 602.

The control unit 606 is configured to receive biometric data from one or more physiological sensors 604. The physiological sensors 604 may include electrocardiogram electrodes, photoplethysmography devices, oscillometric pressure cuffs, temperature probes, and electrochemical biomarker sensors, each configured to detect real-time physiological conditions. The control unit 606 analyzes the incoming data using its AI engine to identify anomalies, select appropriate pharmaceutical compounds, and determine dosing parameters.

The control unit 606 is further configured to transmit dosing commands to subcutaneous delivery modules 608. The subcutaneous delivery modules 608 may include micro-pump units with cartridge interfaces, retractable cannula assemblies, and anti-backflow valves for controlled subcutaneous infusion.

In some embodiments, communication between the physiological sensors 604, the control unit 606, and the subcutaneous delivery modules 608 may occur via Bluetooth Low Energy (BLE) or software-defined radio (SDR) utilizing adaptive frequency hopping. In other embodiments, secure mesh or satellite links may be employed to maintain operational connectivity in contested or remote environments.

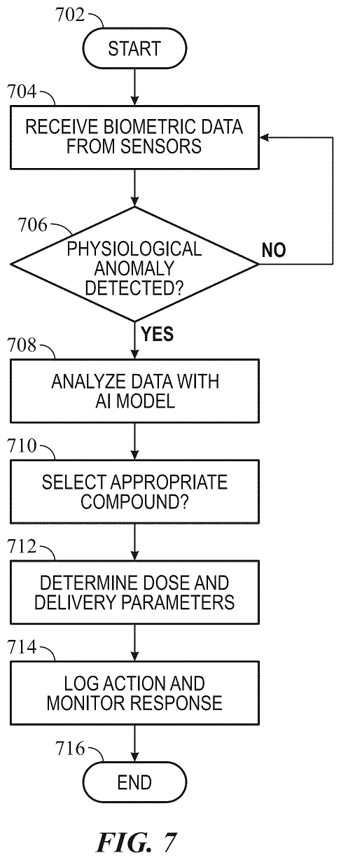

FIG. 7 illustrates a flow diagram of an autonomous decision-making process performed by the modular, wearable battlefield medical system. The process begins at step 702 and proceeds to step 704, where biometric data is received from one or more physiological sensors. The physiological sensors may be configured to monitor parameters such as electrocardiogram signals, blood oxygen saturation, blood pressure, skin temperature, or biochemical markers.

At decision step 706, the system determines whether a physiological anomaly is detected. If no anomaly is detected, the process returns to step 704 to continue monitoring sensor data. If an anomaly is detected, the process proceeds to step 708, where the control unit analyzes the data using an artificial intelligence (AI) model. The AI model may evaluate the biometric data to classify the type of anomaly, such as hemorrhage, dehydration, or arrhythmia.

At decision step 710, the process evaluates whether an appropriate compound for treatment is available within the cartridges of the subcutaneous delivery modules. If no compound is available, the process may trigger a request for remote medical input. If a compound is available, the process advances to step 712, where the system determines a dosage and delivery parameter set, including infusion volume, rate, and timing.

At step 714, the system logs the action in secure storage and monitors the user's response through continued sensor input. The process then concludes at step 716, although monitoring and feedback may return the system to step 704 for continuous closed-loop operation.

In this disclosure, the descriptions of the various embodiments have been presented for purposes of illustration and are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein. Thus, the appended claims should be construed broadly, to include other variants and embodiments, which may be made by those skilled in the art.

It will be appreciated by persons skilled in the art that the present embodiment is not limited to what has been particularly shown and described hereinabove. A variety of modifications and variations are possible considering the above teachings without departing from the following claims.

What is claimed is:

1. A modular wearable medical system comprising:
a plurality of subcutaneous delivery modules each configured for anatomical placement on an upper limb of a user;
each delivery module including a housing affixed via a biocompatible retention structure, a micro-pump, and a cartridge interface configured to receive a replaceable cartridge;
each replaceable cartridge comprising at least one fluid compartment containing a pharmaceutical compound and constructed from a chemically inert, biocompatible material;
a cannula deployment mechanism configured to advance a needle through a skin surface to create an insertion pathway, position a flexible cannula within subcutaneous tissue through the insertion pathway, and retract the needle while leaving the flexible cannula in place for subcutaneous infusion;
a set of physiological sensors configured to detect biometric data including electrocardiogram, peripheral oxygen saturation, oscillometric blood pressure, skin temperature, and at least one biochemical marker;
a control unit including an artificial intelligence engine configured to receive the biometric data, classify a physiological anomaly from among a plurality of anomaly types including hemorrhage, dehydration, and arrhythmia, select a pharmaceutical compound from among the pharmaceutical compounds available in the cartridges of the plurality of subcutaneous delivery modules based on the classified physiological anomaly, determine a compound delivery parameter, and transmit activation instructions to the micro-pump of the corresponding subcutaneous delivery module; and
a communication interface configured to receive remote control signals and transmit biometric and system data to an external device,
wherein the control unit is further configured to monitor a physiological response of the user via the set of physiological sensors following compound delivery and adjust subsequent compound delivery parameters based on the monitored physiological response in a closed-loop manner.

2. The system of claim 1, wherein each replaceable cartridge is configured with a click-lock mechanical coupling to the micro-pump and includes an embedded seal sensor to prevent infusion without proper alignment.

3. The system of claim 1, wherein each micro-pump unit is coated with a conformal protective layer resistant to humidity, chemical infiltration, and microparticles.

4. The system of claim 1, wherein each replaceable cartridge further comprises separate fluid channels for each compartment. electronically controlled valves to prevent cross-contamination.

5. The system of claim 1, wherein the communication interface comprises a software-defined radio configured for adaptive frequency hopping and channel reassignment under spoofing or jamming attempts.

6. The system of claim 1, wherein each micro-pump unit contains a hardware-based trusted platform module for cryptographic operations and tamper protection.

7. The system of claim 1, wherein each replaceable cartridge includes an encrypted NFC chip for authentication prior to activation.

8. The system of claim 1, wherein all components in contact with human tissue comply with ISO 10993 biocompatibility standards.

9.-20. (canceled)
